(19) United States
(12) Patent Application Publication
(10) Pub. No.: US 2011/0137217 A1
(43) Pub. Date: Jun. 9, 2011

(54) METHOD AND DEVICE PRESERVING OF INFANT SLEEP AND AVOIDANCE OF INFANT ADAPTATION

(76) Inventor: Mark Shinnick, Los Angeles, CA (US)

(21) Appl. No.: 12/961,926

(22) Filed: Dec. 7, 2010

Related U.S. Application Data
(60) Provisional application No. 61/267,236, filed on Dec. 7, 2009.

(51) Int. Cl. A61H 1/00; A61N 1/36; A61N 2/00

(52) U.S. Cl. 601/89; 607/2; 607/88; 607/62

(57) ABSTRACT

An apparatus is disclosed for stimulating a sleeping infant, preferably only to the point of slight awakening of the infant, such that the infant's SNS respiratory activation tone is maintained, over a period of time such as days or weeks, in opposition to PNS expression. An enclosure includes a circuit having a power source, a user interface, and timing and random number generator functions for producing successive rest durations, stimulation durations, and stimulation intensities, all based on preset distribution curves with preset variances. At least one stimulating device such, as a mass reciprocally moved by a motor, is electrically interconnected with the circuit. Upon expiration of a rest duration, the stimulating device is activated for the stimulation duration at the stimulation intensity, and then deactivated for the next successive rest duration.

### CROSS-REFERENCE TO RELATED APPLICATIONS

This application claims the benefit of U.S. Provisional Patent Application 61/267,236, filed on Dec. 7, 2009, and which is incorporated in its entirety herein by reference.

### STATEMENT REGARDING FEDERALLY SPONSORED RESEARCH AND DEVELOPMENT

Not Applicable.

### FIELD OF THE INVENTION

This invention relates to stimulation devices, and more particularly to an infant stimulation device and method for use during sleep.

### DISCUSSION OF RELATED ART

Sudden infant death (SIDS) is a serious problem with over 2,500 deaths occurring in the United States each year. It has become better understood in the last few years that there are multiple approaches for reducing the occurrence rate of SIDS. One category of approach has been advice to parents on infant sleeping position and the types of items that might or might not be recommend being in the crib with a sleeping child. Another, more proactive approach has been the development of a variety of schemes for getting an infant to go to sleep. These include devices to rock the baby, play soothing music, etc. They can be characterized a stimulating the Para-sympathetic (“PNS”). A third category concerns itself with alarms that attempt to monitor infant breathing and take some dramatic action to wake up the baby and her caregivers. These devices can be thought of as stimulating the sympathetic (“SNS”) nervous systems.

One view of “baby rocker” type solutions is that they accentuates the infant's PNS system and that an over-dominance of that system can engender infant sleep apnea, thus creating a high risk of inducing common SIDS.

The monitor-alarm-and-wake-up approaches remain wanting because the stimulations would need to be somewhat violent to be of medical value in treating the already-apnic infant. Such practice is further detrimental to sleeping infants because of the significant inconvenience intrinsic to such monitoring systems, especially their many false alarms that result in interruption of infant rest periods that are vital in quickly maturing the infant's nervous system.

There seem to be least two shortcomings in the related art: 1) The risk of baby rockers and bed vibrators inducing and supporting parasympathetic over-dominance in suppression of the sympathetic nervous system's vital signaling of respiratory drive; and 2) conceptual failure to subtly preserve conditions of interim autonomic nervous system balance required for high quality developmental progression attaining matured stability of respiratory activation tone.

What has been needed is an approach that does not overly induce a PNS dominated deep sleep or rely on violent mechanical stimulation when an infant has already stopped breathing.

### SUMMARY

The present teachings involve stimulating a sleeping infant, preferably never further than the point of slight awakening, such that the infant's SNS respiratory activation tone is maintained, over a period of time, in opposition to excessive PNS expression. Embodiments can include a timer providing a varying time interval that operatively coupled to a system for applying a moderate degree of a physical stimulus to an infant's sleeping environment. In some versions the time interval is pseudorandom. The center and standard deviation of the timer can be user adjustable in some versions. In some versions the characteristics of the pseudorandom timing can be dynamically affected by factors such as the infant's rate of breathing. In some versions the varying time intervals can be predetermined, possibly in the form of a table of data.

The frequency, duration and intensity of the stimulations can be such as to maintain the infant's nervous system below the threshold of full arousal from his normal resting state. Stimulation modalities can include, for example, a mass oscillating means such as a mass reciprocally moved by a motor to produce a vibration in the enclosure and any object that contacts the enclosure, such as a crib mattress or the like. The stimulating modality can be airflow, illumination, or a sound, for additional examples. In some embodiments multiple varying types of stimulation generators can be used, possibly alternating or by a random selection. Other stimulation sources may be utilized provided they have the effect of being able to slightly awaken the infant when activated.

Upon expiration of a timer's rest duration, a circuit can activate a stimulation source for a stimulation duration at a stimulation intensity. As such, the infant is stimulated periodically or sporadically while sleeping. The stimulation intensity may be a substantially random intensity. In one embodiment of the method, the average and standard deviation of the rest durations are preset according to observed apnea exacerbation of the infant. Also the average and standard deviation of the rest durations can be preset according to observed sleep cycle durations of the infant in some versions.

These teachings include the possible use of an audio or audio/video recording means or an accelerometer for recording the infant's response. Some versions can include a data port for allowing an external device to read data from, or possibly control, the operation of an apparatus consistent with these teachings.

### DESCRIPTION OF THE DRAWINGS

FIG. 1 is a perspective view of an embodiment of the invention as mounted to a crib with a sleeping infant;

FIG. 2 is a cross-sectional view of the apparatus of FIG. 1, taken generally along lines 2-2 of FIG. 1;

FIG. 3 is a perspective exploded view of the apparatus of FIG. 1, illustrating an enclosure, an AC adapter, and a selection of stimulation source thereof;

FIG. 4 is a graph showing a hypothetical situation of device in use, illustrating a stimulation intensity and duration between rest durations;

FIG. 5 is a chart showing a list of stimulation and rest durations, and stimulation intensities;

FIG. 6 is an example of a preset stimulation duration distribution curve;

FIG. 7 is an example of a preset rest duration distribution curve;

FIG. 8 is an example of a preset stimulation intensity distribution curve;

FIG. 9 is a functional diagram of an alternate mass oscillating means; and

FIG. 10 is the alternate mass oscillating means of FIG. 9.

### DETAILED DESCRIPTION

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. Further, the term “random” herein may refer to pseudorandom events, such as those generated by a so-called random number generating electronic circuit or computer algorithm, even though such numbers are not truly random.

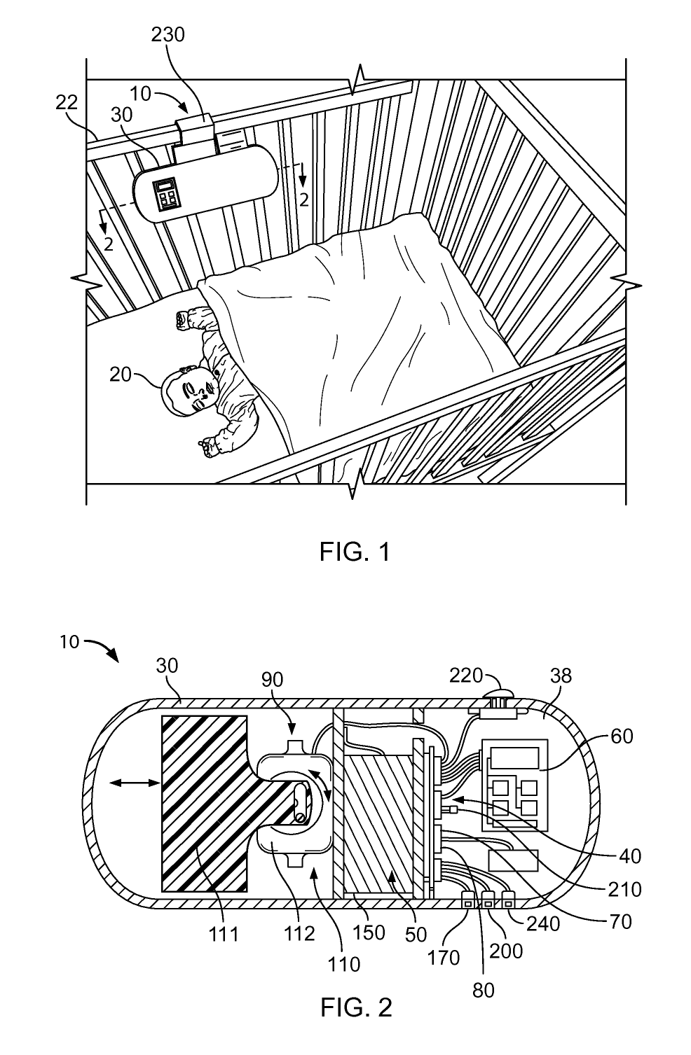

FIGS. 1 and 2 illustrate an apparatus for stimulating a sleeping infant 20, preferably only to the point of slight awakening of the infant 20, such that the infant's SNS respiratory activation tone or balance is maintained.

FIGS. 1 and 2 illustrate an enclosure 30, preferably made from plastic material, that has at least one hollow portion 38 therein. An attachment means 230 may be included for selectively fixing the enclosure 30 to an object, such as a crib 22. Such an attachment means 230 may be at least one hook-and-loop type fastening strap, a belt with a buckle or mechanical snaps, mechanical clamps, other mechanical fasteners, adhesive tape, or the like.

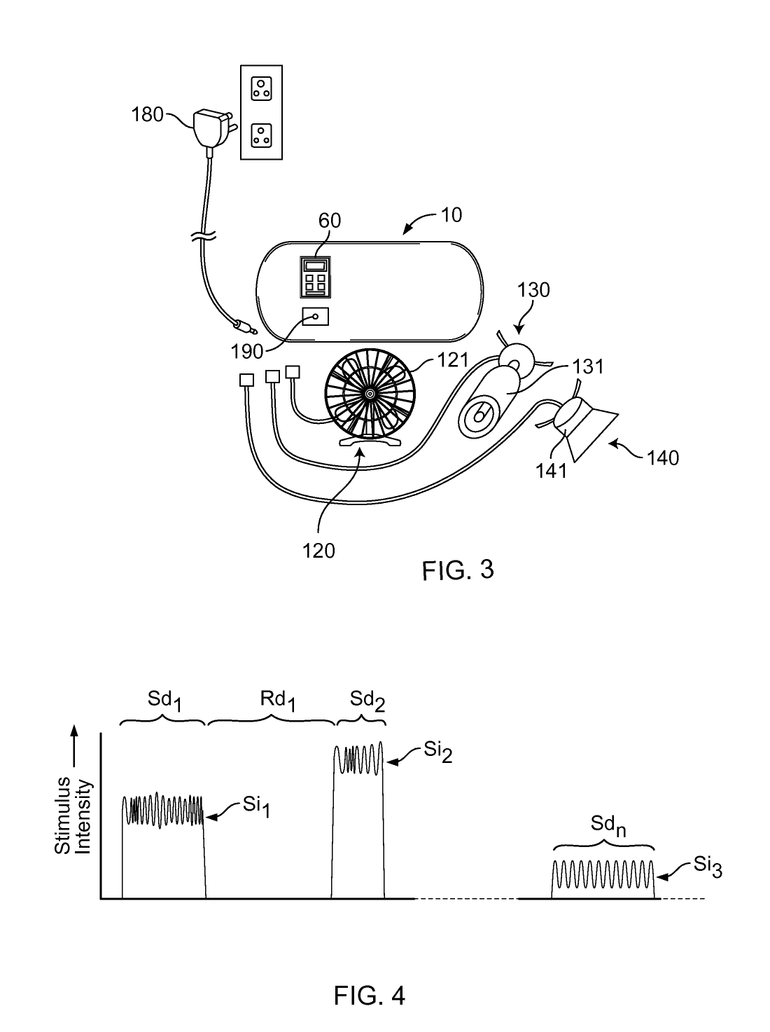

A circuit 40 is contained at least partially within the enclosure 30 and includes a power source 50, a user interface 60, a timer 70, and an interval offset generator 80. The power source 50 may be a rechargeable battery 150, for example, with an AC adapter port 170 electrically interconnected thereto and accessible through the enclosure 30 and adapted to electrically interconnect with and be recharged by an external AC adapter 180 (FIG. 3). A switch 220, such as a manual switch, an electronically toggled switch, or the like, may be included for selectively activating or deactivating the apparatus 10.

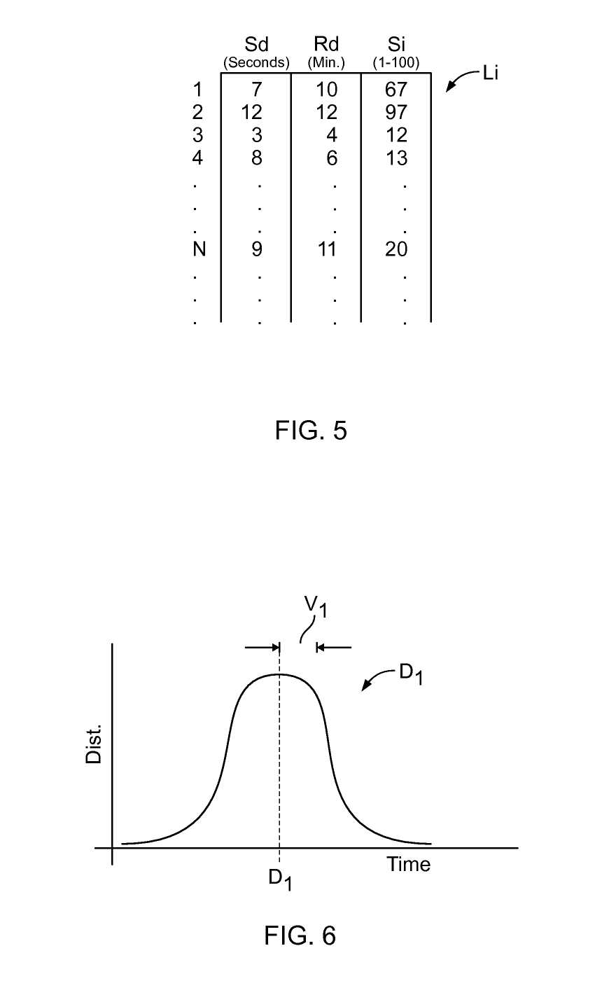

The interval offset generator 80 may be a random offset interval generator 100, whereby at least each successive rest duration RDₙ₊₁ varies from a previous rest duration RDₙ according to a first preset random distribution D1 and a first standard deviation V1 (FIGS. 4 and 6). That is, the timer 70 sets the average value of distribution D1, and the office interval generator 100 determines a plus-or-minus interval offset I/Oₙ₊₁ applied to each successive rest duration RDₙ₊₁(FIG. 5).

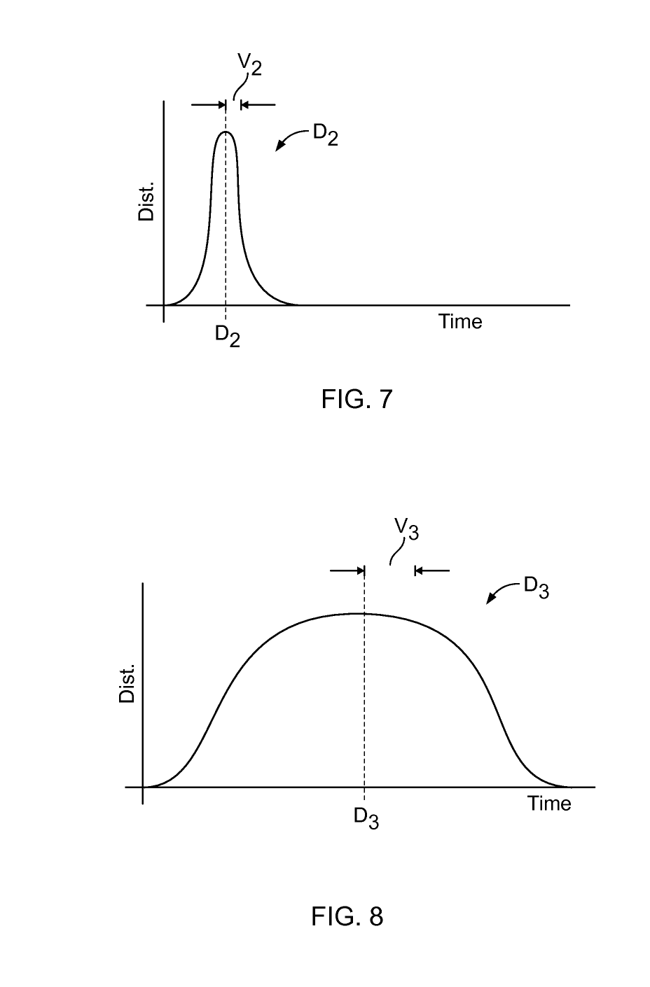

Each successive stimulation duration SDₙ₊₁ may also vary from the previous stimulation duration SDₙ according to a second preset random distribution D2 and a second standard deviation V2 (FIG. 7), also implemented by the timer 70 and the interval offset generator 80. Such a timer 70 and an interval offset generator 80 may be enacted with timing circuits, random number generator routines, a CPU, a memory, and a set of software instructions common in the art.

Alternately, the interval offset generator 80 may be a list LI of rest durations RDₙ, whereby at least each successive rest duration RDₙ₊₁ varies from the previous rest duration RDₙ according to the list LI (FIG. 5). Likewise, each stimulation duration SD and stimulation intensity SI is set according to the list LI. Such a list LI may be preset is during manufacture, for example, or programmed into a memory of the circuit 40 once deployed, such programming being based on the observed sleeping characteristics of the infant 20. For example, the interval offset generator 80 may be driven by accelerometer-derived extrapolation of breathing rate trend prediction of apnea, as determined by the circuit and an accelerometer 210 fixed within the enclosure 30. The breathing rate of the infant 20 is determined based on accelerometer readings, or other sensor readings as necessary, and as the breathing rate of the infant 20 slows or otherwise indicates a trend towards apnea in the infant 20, the interval offset generator 80 may “time out” or otherwise signal the circuit 40 that the rest duration RDₙ has ended.

Alternately, the timer 70 and the interval offset generator 80 may be combined in a random number generating routine that has as inputs, for example for the rest durations RD, of the desired average value of D1 and the variance V1.

At least one stimulation source 90 is electrically interconnected with the circuit 40. Such a stimulation source 90 may be, for example, a mass oscillating means 110 such as a mass 111 reciprocally moved by a motor 112 (FIG. 2) to produce a vibration in the enclosure 30 and any object that contacts the enclosure 30, such as a crib mattress or the like. The mass may be from 600 to 1000 grams, for example. The motor 112 may run at between one and five revolutions-per-second, for example, such that the mass 111 oscillates at a frequency of between one-half and nine hertz. The stimulation source 90 may alternately include the mass 111 and a reciprocating linear actuator (not shown), or the like. Alternately, the stimulation source 90 may be an air blowing means 120, such as a fan 121 for stimulating the infant 20 with wind at about 10 CFM, for example. Alternately, the stimulation source 90 may be an illumination means 130, such as a lamp 131, for stimulating the infant 20 by visible light at between 40 W and 120 W from a distance of 1 to three feet, for example. Alternately, the stimulation source 90 may be a sound producing means 140, such as a speaker 141, for stimulating the infant 20 by audible sound at between 70 db and 90 db. The stimulation source 90 may be external to the enclosure 30 and selectively chosen, for example, by plugging the stimulation source 90 into an electronic accessories port 240 electrically interconnected with the circuit 40 (FIG. 2). In the case where multiple varying types of stimulation source 90 are connected to the circuit 40, the circuit 40 can vary which stimulation source 90 is activated at any given time, such as by alternating or by randomly selecting via a random selection function of the circuit 40. Other stimulation source 90 may be utilized provided they have the effect of being able to slightly awaken the infant 20 when activated.

In use, with the enclosure 30 supported proximate the sleeping infant 20 and arranged so that the mass 111 oscillates in a direction orthogonal to the longitudinal axis of the infant 20, for example, a next rest duration RDₙ is determined with the timer 70 and the interval offset generator 80. Upon expiration of the rest duration RDₙ, the circuit 40 activates the stimulation source 90 for a stimulation duration SDₙ at a stimulation intensity SIₙ, determining the next successive rest duration SDₙ₊₁, and repeating the process until the apparatus 10 is deactivated. As such, the infant 20 is stimulated periodically while sleeping, each successive rest duration RDₙ₊₁ varying according to the interval offset generator 80. The stimulation intensity SI may be a substantially random intensity, is whereby at least each successive stimulation intensity SIₙ₊₁ varies from the previous stimulation intensity SIₙ by a substantially random level according to a third preset random distribution D3 and a third standard deviation V3 (FIG. 8). In one embodiment of the method, the average and standard deviation of the rest durations RD are adjusted according to observed apnea exacerbation of the infant 20. Alternately, the average and standard deviation of the rest durations RD are adjusted according to observed sleep cycle durations of the infant 20.

The circuit 40 may further include an audio or audio/video or accelerometer data recording means 190 for recording the infant's response, regardless of when the stimulation source 90 is activated, or for detecting if a particular stimulation intensity SI or stimulation duration SD is excessive and results in waking the infant 20 too completely. The circuit 40, or a medical professional, for example, may then determine a maximum SI and/or SD. The circuit 40 further includes a data port 200 electrically interconnected to the circuit 40 and accessible through the enclosure for allowing an external device (not shown) to read data previously recorded by the recording means 190. Such data may be used to alter the random distributions D1, D2, D3 and standard deviations V1, V2, V3 for example, as determined by a medical professional. Similarly, a heart rate and breathing monitoring means (not shown) may also be included for providing similar data for a medical professional's evaluation.

The circuit 40 may also include a transmission and control means (not shown), so that the apparatus 10 may be controlled remotely, and for transmitting to a remote device (not shown), such as a so-called “smart phone.” As such, the infant's response to the stimulation source 90 as recorded by the audio or audio/video recording means 190, or the heart rate and breathing monitoring means, may be transmitted remotely to a parent or doctor, for example, through the remote device. Further, the remote device may control one or more of the apparatus 10, such as two apparatuses 10 placed on either side of the infant 20 to form a cradle structure (not shown). Each of the apparatuses 10 in such a configuration are controlled by the remote device, and each apparatus 10 may stimulate the infant 20 in-phase with each other in some instances, out of phase in other instances, and each at times independently, for example, as part of a strategy to prevention infant adaptation. Each apparatus 10 may start a stimulation duration SDₙ independently from that of the other apparatus 10, for example. Either the remote device or one of the apparatus 10 designated as the “master” device, may control such a stimulation program of multiple apparatuses 10. Each apparatus 10 may communicate with the others wirelessly, as is known in the art, or through a cable arrangement (not shown) that is elevated above the infant 20, or positioned under the infant's mattress for example, so that the infant 20 has reduced risk of becoming entangled thereby.

Further, circuit 40 may also include the accelerometer 210 so that logic in the circuit 40 may determine an oscillation frequency of the mass 111 that results in the greatest resonance of the enclosure 30 during the stimulation duration SD. The circuit 40 may also be electrically connected to a motion sensing device, such as an infrared motion sensor (not shown), for detecting movement of the infant 20. Such movement may be indicative of the infant 20 waking from sleep, for example, in which case the circuit 40 may lower the stimulation intensity of each stimulation source 90, or deactivate same. Alternately the motion sensing device may be a breathing rate detector, a heart rate detector, or the like. Such a breathing rate detector or heart rate detector may be in the form of a sensor disk (not shown) temporarily attachable to the infant's clothing proximate the infant's chest, for instance.

The stimulation durations SD can have a mean value of between 10 and 60 seconds, preferably between rest durations RD of from ⅓ to ⅔ of the infant's nominal sleep cycle duration. The apparatus 10 can be programmed or set to infer upon the infant's mentality that his presence is being accommodated, but only incidentally so, within a safe environment otherwise containing intelligent activity and stimuli of various types.

One mode of the invention performs the method of programmed differential modes of non-invasive stimulations to the sleeping infant 20 at a variable interval frequency (SD+RD) of no less than one-half of a infant's mean sleep cycle duration, where stimulations are effected by means of a variation of programmed: i.) 0.5-9 Hz frequency rhythmic lateral physical bedding surface vibrational motion with the mass oscillating means 110; ii.) air flow/evaporative cooling upon the skin with the air blowing means 120; iii.) 0.5-9 Hz event frequency lighting with the illumination means 130, and; iv.) aural and sonic percussive stimulations with the sound producing means 140. A variable combination of these stimulations need only approximate physical energies as were heretofore relatively unappreciated for intrinsic importance to the sleeping infant 20, but are commonly is unwittingly produced by a caregiver's gentle handlings. Such stimulation is further defined as being within the scope of: a.) random vectored reciprocating lateral bedding surface excursions generally less than 50 mm, with rates of motion below 9 HZ that are non-waking, b.) air velocities upon the infant's skin no greater than that from exhaled breathing of a caregiver, for example, c.) light intensity no greater than 35,000 lux, and; d.) sound pressures no greater than 60 decibels. The apparatus 10 further may vary its programmed differential modes of stimulations through alterations in: a.) periods between stimulations (RD), b.) Stimulation waveform pattern and duration (SD), and c.) energies of stimulations (SI), which are all randomized as to their timing and strength as a means of anticipating possible infant adaptation. Consequently, within all the above parameters that define this method, the apparatus 10 functions to deliver to the sleeping infant 20 at variable times, one or more programmed differential stimulations at varying rates, amplitudes, and energies.

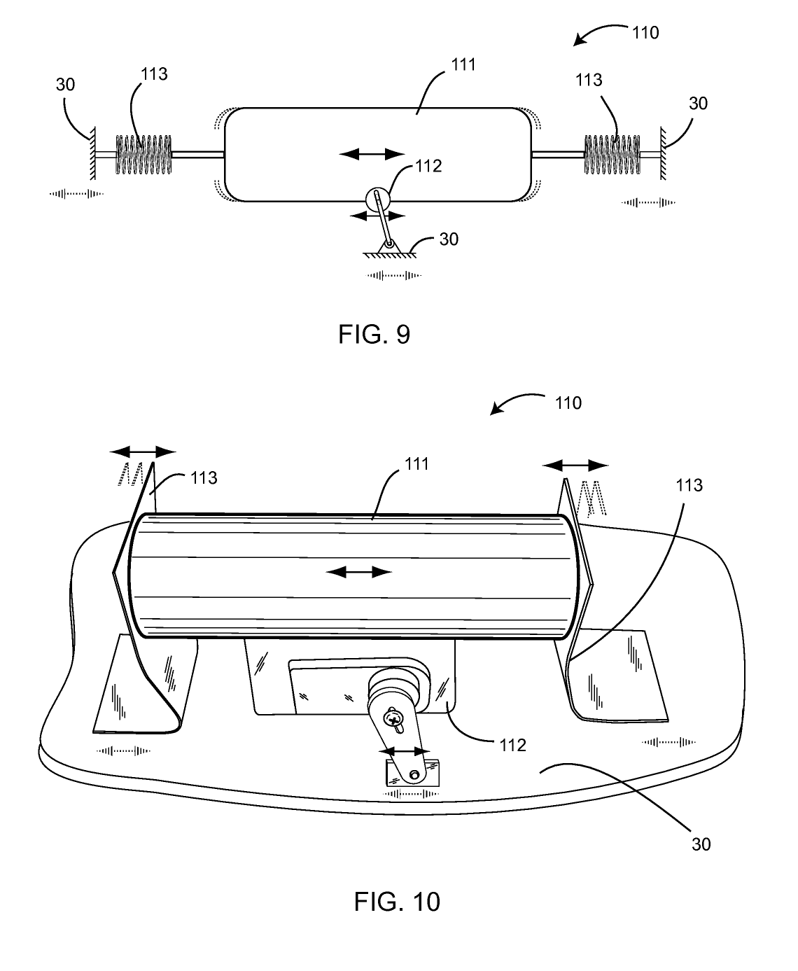

In one embodiment of the invention, the mass oscillating means 110 takes the form of the mass 111 reciprocally moved by a motor 112 (FIGS. 9 and 10) fixed thereto, the mass 111 held vertically above a base of the enclosure 30 by two resilient standoffs 113, such as fiberglass or other suitably resilient materials. The motor 112 may run at between one-half and five oscillations-per-second, for example. The two resilient fiberglass standoffs 113 each act partially as a spring to help the motor 112 return the mass 111 upon motor reversal.

While a particular form of the invention has been illustrated and described, it will be apparent that various modifications can be made without departing from the spirit and scope of the invention. For example, the enclosure 30 may be incorporated into a crib mattress, or the crib 22 itself. The apparatus and method has further application to adult and elder life/sleep quality improvements and anxiety reduction, and application in the fields of human performance assurance; sleep study and control, adult sleep aids, child protective quality assurance, and animal care. Further, the mass oscillating means 110 may be any suitable, relatively quiet mechanical means for reciprocating the mass 111. Accordingly, it is not intended that the invention be limited, except as by the appended claims.

The teachings provided herein can be applied to other systems, not necessarily the system described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above Detailed Description. While the above description details certain embodiments of the invention and describes the best mode contemplated, no matter how detailed the above appears in text, the invention can be practiced in many ways. Details of the system may vary considerably in its implementation details, while still being encompassed by the invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

While certain aspects of the invention are presented below in certain claim forms, the is inventor contemplates the various aspects of the invention in any number of claim forms. Accordingly, the inventor reserves the right to add additional claims after filing the application to pursue such additional claim forms for other aspects of the invention.

What is claimed is:

1. An apparatus for stimulating a sleeping infant, comprising:
a circuit having at least a user interface, a timer, and an interval offset generator; and
a stimulation source operatively coupled to the circuit;
whereby upon expiration of a rest duration determined by the timer and the interval offset generator, the circuit temporarily activates the stimulation source for a stimulation duration at a stimulation intensity and then repeats, stimulating the infant periodically while the infant sleeps, each successive rest duration being determined according to the interval offset generator.

2. The apparatus of claim 1 further including an enclosure having at least one hollow portion therein, the circuit contained at least partially within the enclosure.

3. The apparatus of claim 1 wherein the interval offset generator is a random offset interval generator, whereby each successive rest duration varies from the previous rest duration by a substantially random interval according to a first preset random distribution and first standard deviation.

4. The apparatus of claim 1 wherein at least each successive stimulation duration also varies from the previous stimulation duration by a substantially random interval according to a second preset random distribution and second standard deviation.

5. The apparatus of claim 3 wherein at least each successive stimulation duration also varies from the previous stimulation duration by a substantially random interval according to a second preset random distribution and second standard deviation.

6. The apparatus of claim 1 wherein the interval offset generator is a list of preset offset intervals, whereby at least each successive rest duration varies from the previous rest duration by the next successive offset interval according to the list of preset offset intervals.

7. The apparatus of claim 1 wherein the stimulation intensity is a substantially random intensity, whereby at least each successive stimulation intensity varies from the previous stimulation intensity by a substantially random level according to a third preset random distribution and third standard deviation.

8. The apparatus of claim 1 wherein the stimulation source is a mass oscillating means, whereby the infant is stimulated by tactile vibration.

9. The apparatus of claim 2 wherein the stimulation source is the mass oscillating means contained within the enclosure, whereby the infant is stimulated by tactile vibration.

10. The apparatus of claim 1 wherein the stimulation source is an air blowing means, whereby the infant is stimulated by wind.

11. The apparatus of claim 1 wherein the stimulation source is an illumination means, whereby the infant is stimulated by visible light.

12. The apparatus of claim 1 wherein the stimulation source is a sound producing means, whereby the infant is stimulated by sound.

13. The apparatus of claim 1 wherein the circuit further includes an audio or audio/video recording means for recording the infant's response when the stimulation source is activated, the circuit further including a data port electrically interconnected to the circuit for accessing the audio/video recordings electronically.

14. The apparatus of claim 8 wherein the circuit further includes an accelerometer, whereby the circuit may determine an oscillation frequency of the mass that results in the greatest resonance of the apparatus.

15. The apparatus of claim 1 wherein the circuit further includes the accelerometer, the accelerometer adapted to measure infant breathing rate, the circuit adjusting the interval offset generator accordingly.

16. The apparatus of claim 1 wherein the circuit further includes a heart rate sensor adapted to measure the heart rate of the infant, the circuit adjusting the interval offset generator accordingly.

17. A method for stimulating a sleeping infant periodically, comprising the steps:
a) providing a circuit having a user interface, a timer, and an interval offset generator; and a stimulation source operatively coupled with the circuit;
b) instructing that the circuit be supported proximate the sleeping infant;
c) determining a next rest duration with the timer and the interval offset generator;
d) upon expiration of the rest duration, activating the stimulation source for a stimulation duration; and
e) repeating from step c) until the infant wakes.

18. The method of claim 17 wherein step d) is: upon expiration of the rest duration, activating the stimulation source for a pseudorandom stimulation duration.

19. The method of claim 17 wherein step d) is: upon expiration of the rest duration, activating the stimulation source for a pseudorandom stimulation duration at a pseudorandom stimulation intensity.

20. The method of claim 17 wherein the average and standard deviation of the rest durations are adjusted according to observed apnea exacerbation of the infant.

21. The method of claim 17 wherein the average and standard deviation of the rest durations are adjusted according to observed sleep cycle durations of the infant.
